(19) United States
(12) Patent Application Publication
(10) Pub. No.: US 2012/0213672 A1
(43) Pub. Date: Aug. 23, 2012

(54) APPARATUS FOR USE WITH STERILANT VAPOUR GENERATORS

(75) Inventors: Nicholas Mark Turner Adams, Clanville (GB); Oliver Richard Cumberlege, Andover (GB)

(73) Assignee: BIOQUELL UK LIMITED, Andover, Hampshire (GB)

(21) Appl. No.: 13/504,427

(22) PCT Filed: Sep. 17, 2010
(86) PCT No.: PCT/GB2010/001745

§ 371 Date: Apr. 26, 2012

(30) Foreign Application Priority Data
Oct. 30, 2009 (GB) 0919131.3

(51) Int. Cl. A61L 2/20

(52) U.S. Cl. 422/124

(57) ABSTRACT

This invention relates to apparatus for use with a sterilant vapour generator used for sterilising an enclosure, said apparatus providing means for enhancing the distribution of the sterilant vapour in the enclosure during a gassing phase of the sterilisation process and for removing the sterilant vapour from the air in the enclosure during a subsequent aeration phase. The apparatus can optionally be configured to provide humidification, if required. The invention therefore comprises means for the distribution of sterilant vapour in the enclosure during a gassing phase of the sterilisation process and means for removing the sterilant from the air in the enclosure during an aeration phase.

This invention relates to apparatus for use with a sterilant vapour generator used for sterilising an enclosure, said apparatus providing means for enhancing the distribution of the sterilant vapour in the enclosure during a gassing phase of the sterilisation process and for removing the sterilant vapour from the air in the enclosure during a subsequent aeration phase. The apparatus can optionally be configured to provide humidification, if required.

The invention provides an apparatus, for use with apparatus for delivering sterilant vapour to an enclosure for a sterilisation process, said apparatus comprising means for the distribution of sterilant vapour in the enclosure during a gassing phase of the sterilisation process and means for removing the sterilant from the air in the enclosure during an aeration phase.

The distribution means preferably comprise means for creating an air flow in the enclosure.

More preferably the distribution means comprise at least one inlet, at least one outlet and the means for creating an air flow comprise means for drawing air into the apparatus from outside the apparatus through the at least one inlet and discharging the air through the at least one outlet at an increased velocity.

The apparatus may be used in conjunction with a sterilant vapour (e.g. Hydrogen Peroxide) generator to bio-decontaminate enclosures, for example rooms, in which case the apparatus may be located within the space to be decontaminated.

The apparatus may be controlled by the sterilant vapour generator and may be operational in all phases of the bio-decontamination cycle of the generator. During a conditioning phase (and potentially gassing phases) it may provide water vapour to the target enclosure to increase the humidity level; during gassing phases it assists in the vapour distribution; and during an aeration phase it provides the means for removing the sterilant vapour from the air in the enclosure.

The sterilant removal means preferably comprise at least one inlet, means for removing sterilant vapour from the air, at least one outlet, and air moving means for drawing air into the apparatus through the inlets through the sterilant removal means and discharging the air through the outlets to thereby reduce the concentration of sterilant in the air outside the apparatus.

Current commercially available room aerators used in combination with sterilant vapour generators are operational only at the end of the gassing phase of a sterilisation cycle and are typically connected to, and controlled by, gas generators. The apparatus of the present invention is advantageous in that it is also used to assist in the vapour distribution during a bio-decontamination cycle to ensure even vapour distribution through complex/large facilities to negate the need for additional generators or fans.

To perform the aeration function the apparatus may have one or more inlets to receive sterilant containing air from the enclosure (following sterilisation of that enclosure), one or more outlets to return air from which sterilant has been reduced, a catalyst, or other suitable means, within the apparatus for removing sterilant from the air, means for drawing sterilant containing air from the inlet through the sterilant removal means and thence to atmosphere and means to circulate air containing sterilant from the enclosure during a enclosure sterilisation process through that part of the enclosure which is downstream of the filter, or other sterilant removal means, to ensure sterility of that part of the enclosure for the sterilant removal phase.

The apparatus is preferably a single unit which is mounted on wheels to be readily mobile. The apparatus may include means for physical connection to other items of apparatus for ease of transportability.

The apparatus may be left running, i.e. operational, before or at the end of the cycle without still being attached/connected (by wire or wireless) to the generator.

Embodiments of the invention will now be described, by way of example only, reference being made to the accompanying drawings in which:

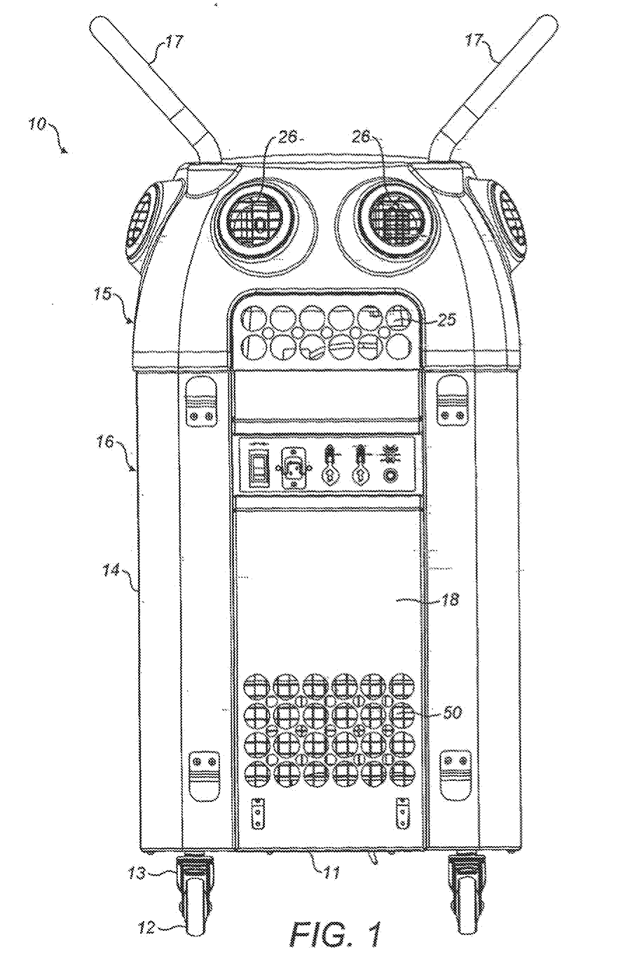

FIG. 1 is a front elevation view of a first embodiment of an apparatus of the present invention;

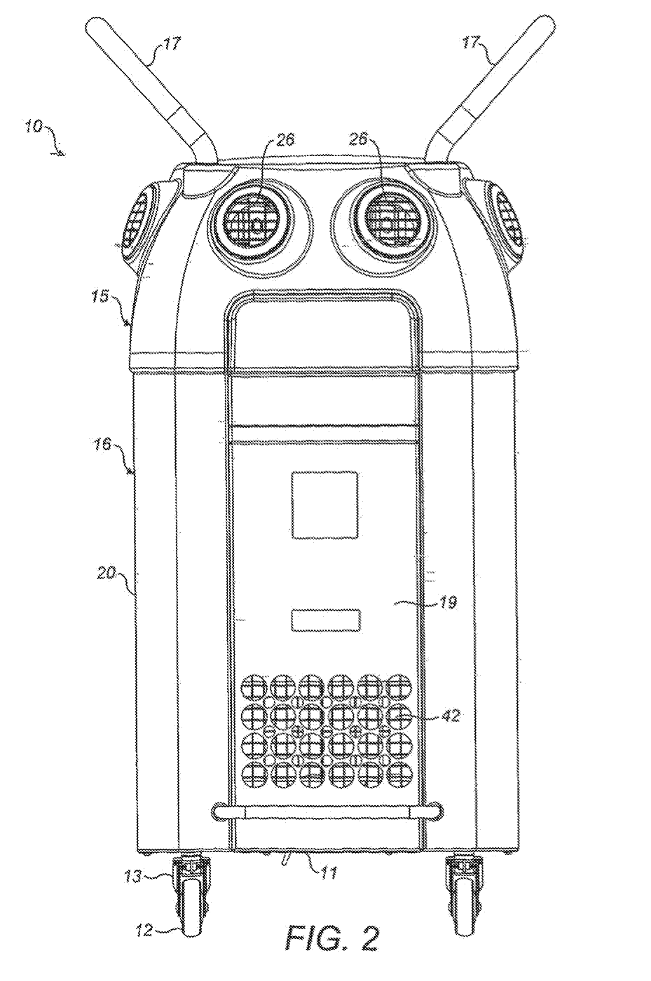

FIG. 2 is a rear elevation view of the apparatus shown in FIG. 1;

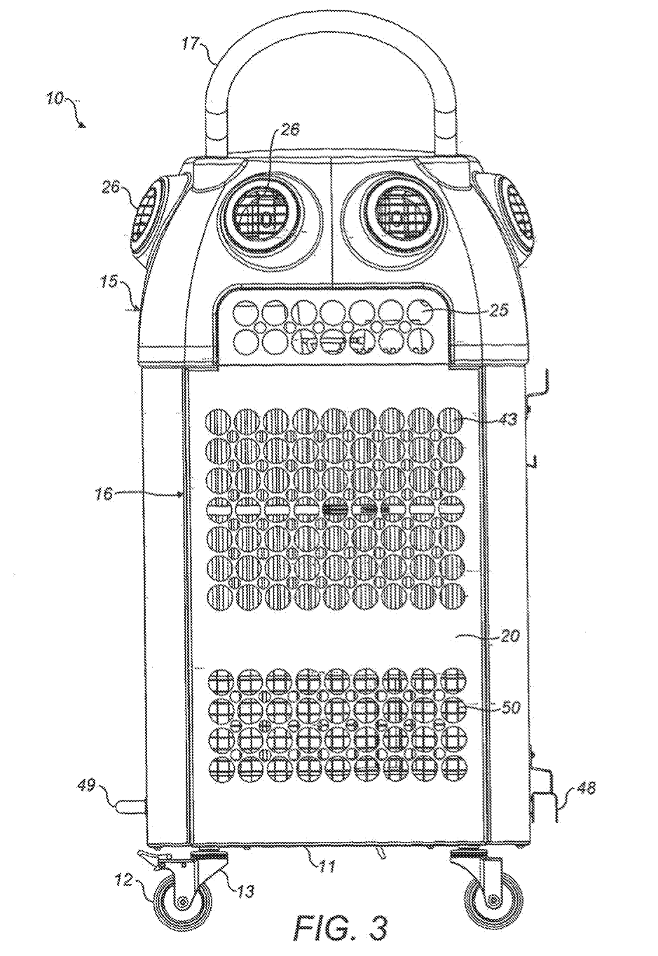

FIG. 3 is a side elevation view of the apparatus;

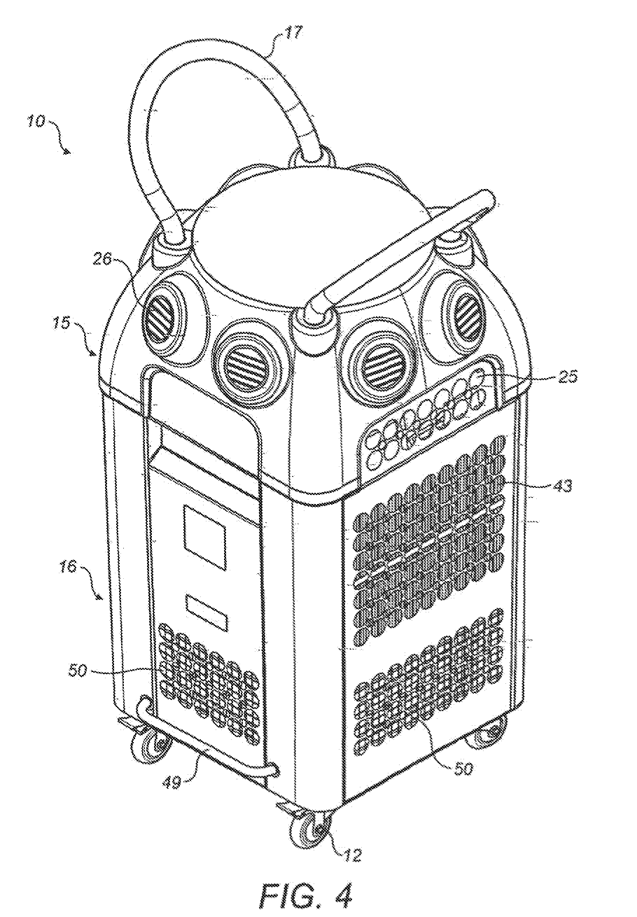

FIG. 4 is a perspective of the apparatus;

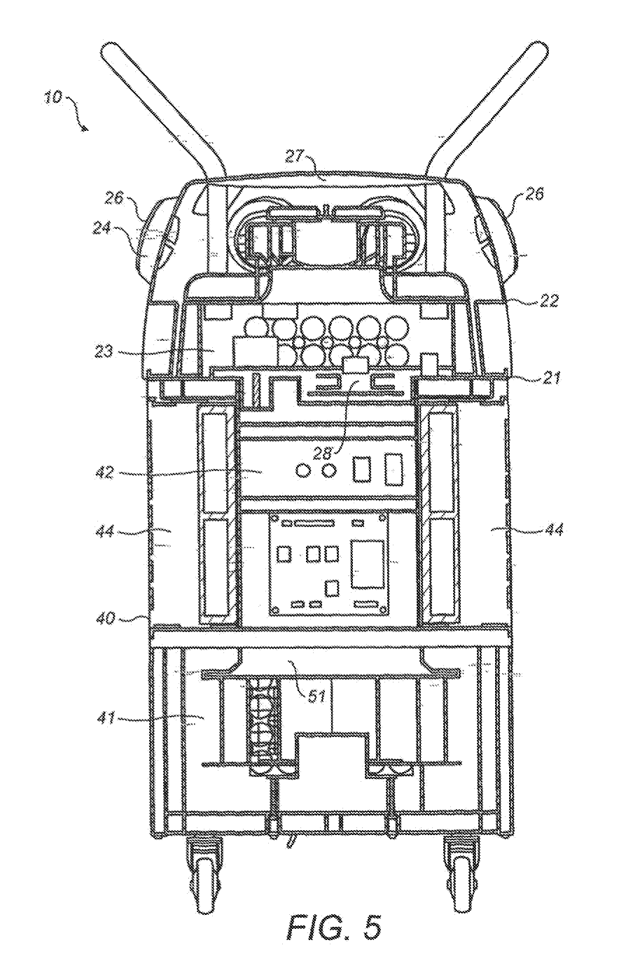

FIG. 5 is a side to side cross-sectional view through the apparatus of FIG. 1;

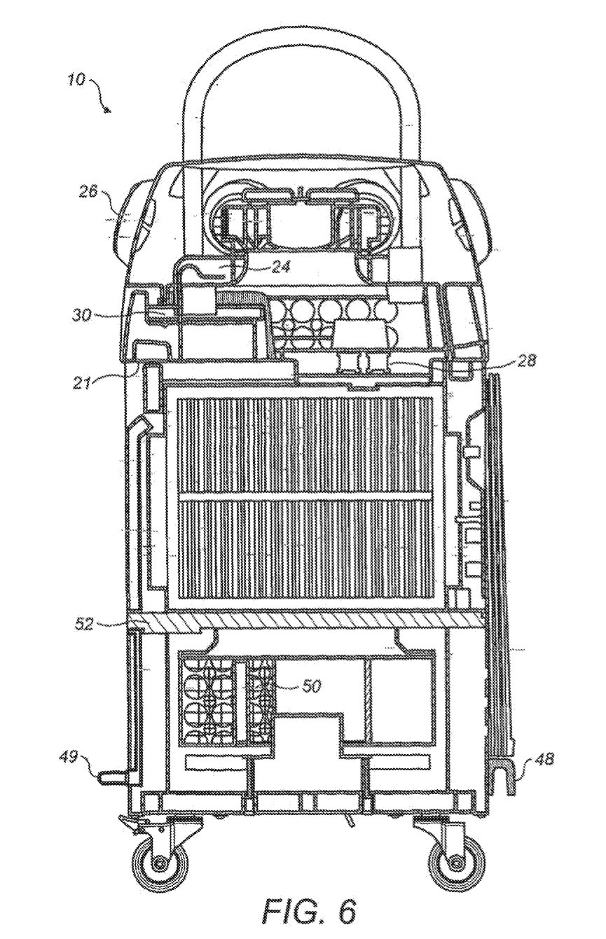

FIG. 6 is a front to back cross-sectional view of the apparatus of FIG. 1;

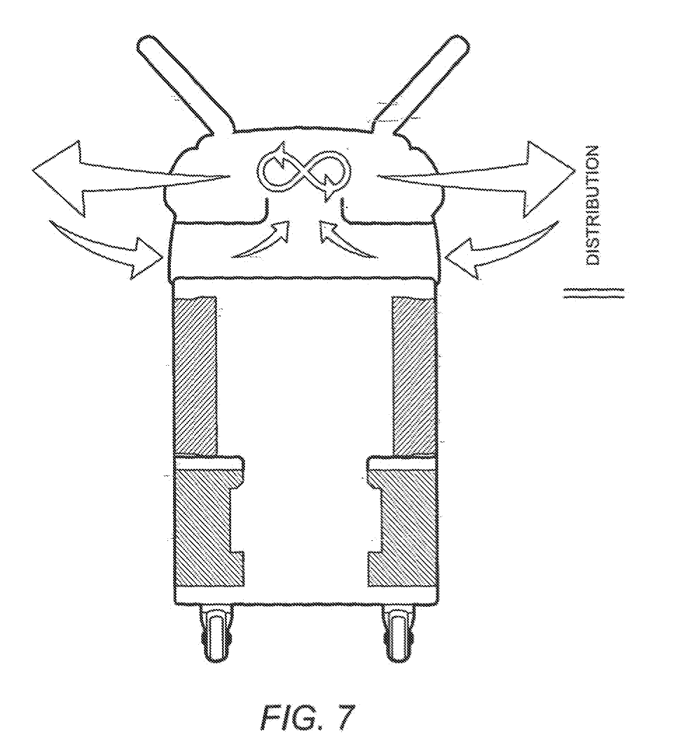

FIG. 7 is a diagrammatic view of the apparatus operating in its distribution mode;

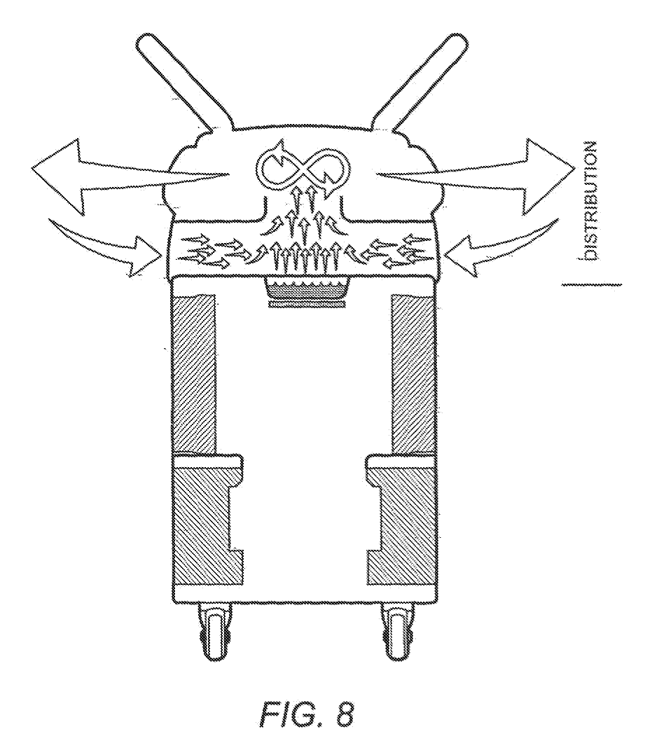

FIG. 8 is a similar view to FIG. 7 with a humidifier function to add vapour to the circulating gas/sterilant vapour;

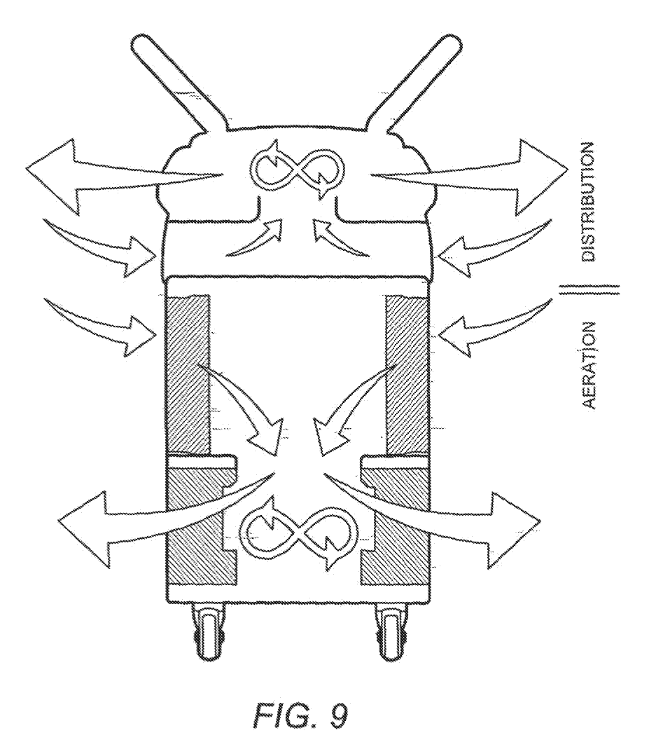

FIG. 9 is a similar view to FIG. 7 with a gas removal or aeration function in which sterilant vapour is removed from the encircling atmosphere;

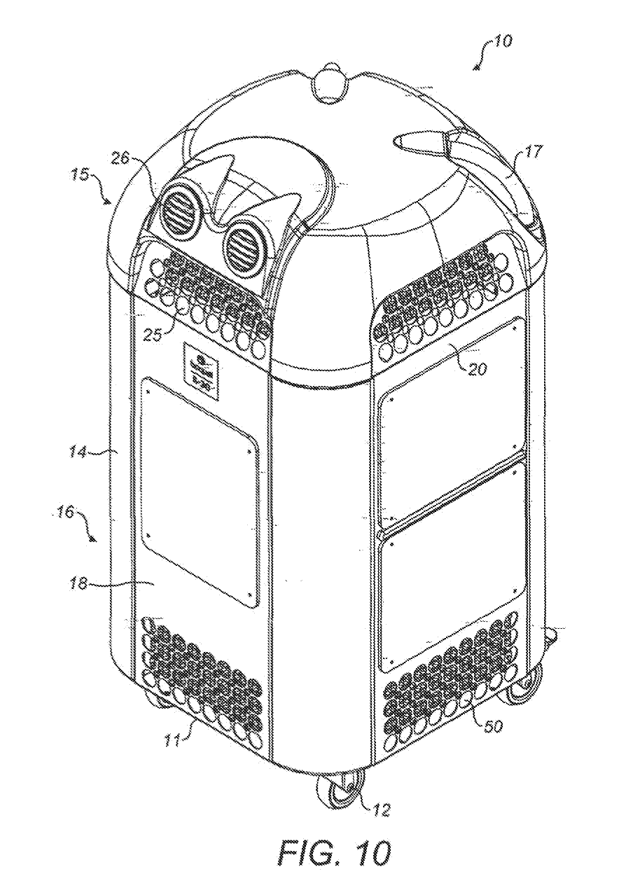

FIG. 10 is a perspective view of a front and side of another embodiment of the apparatus of the present invention;

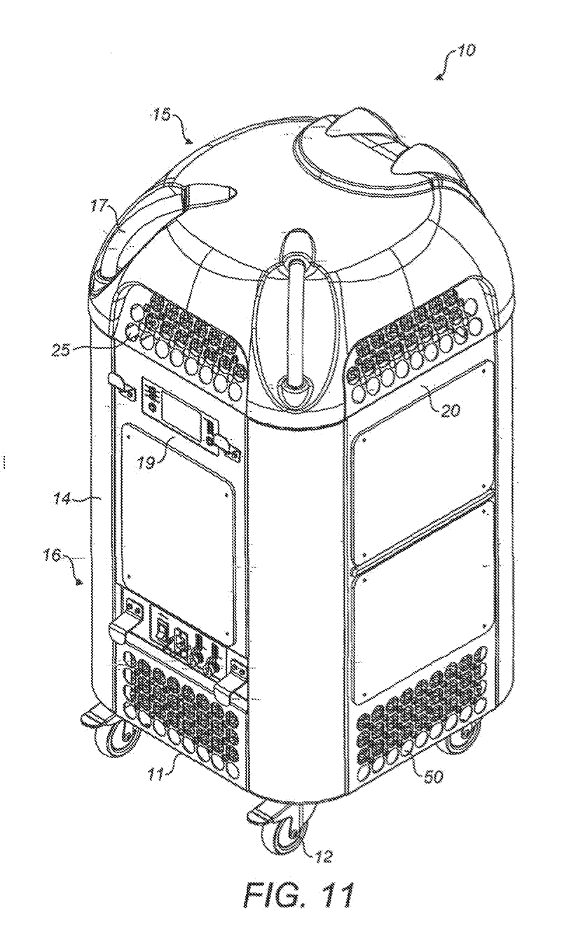

FIG. 11 is a perspective view of a rear and side of another embodiment of the apparatus of FIG. 10;

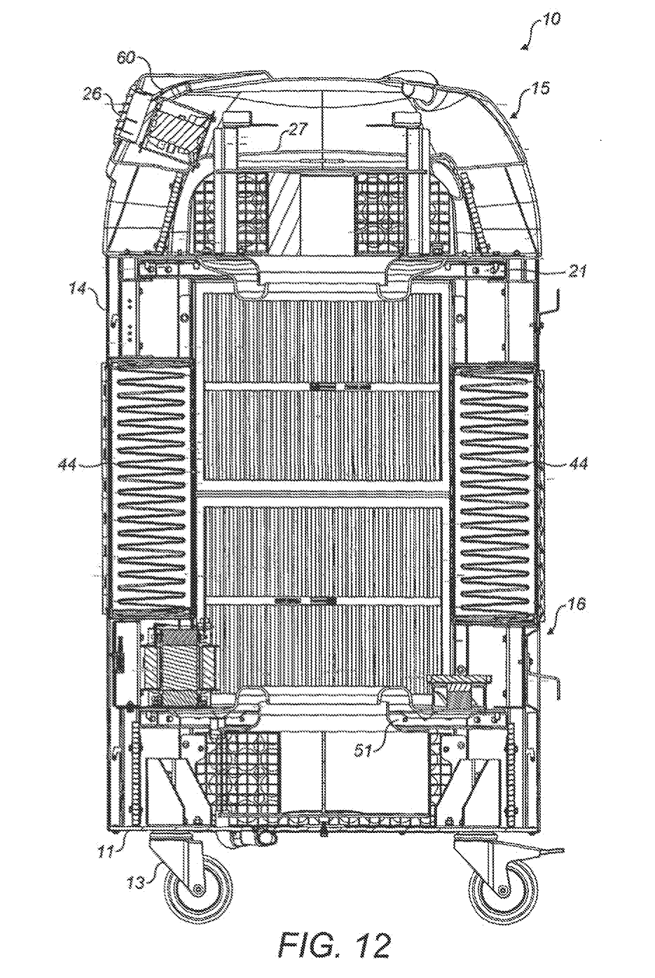

FIG. 12 is a side sectional elevation of the apparatus of FIG. 10;

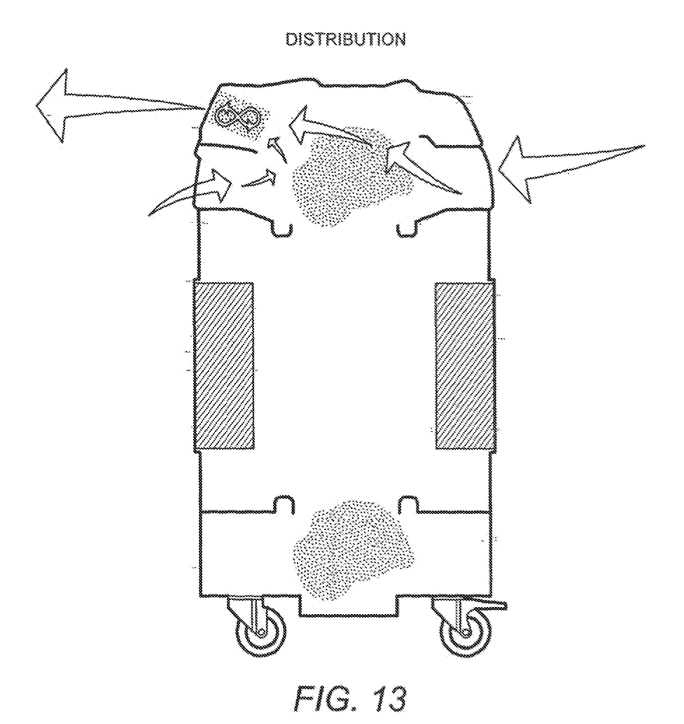

FIG. 13 is a diagrammatic view of the apparatus of FIG. 10 operating in its distribution mode; and

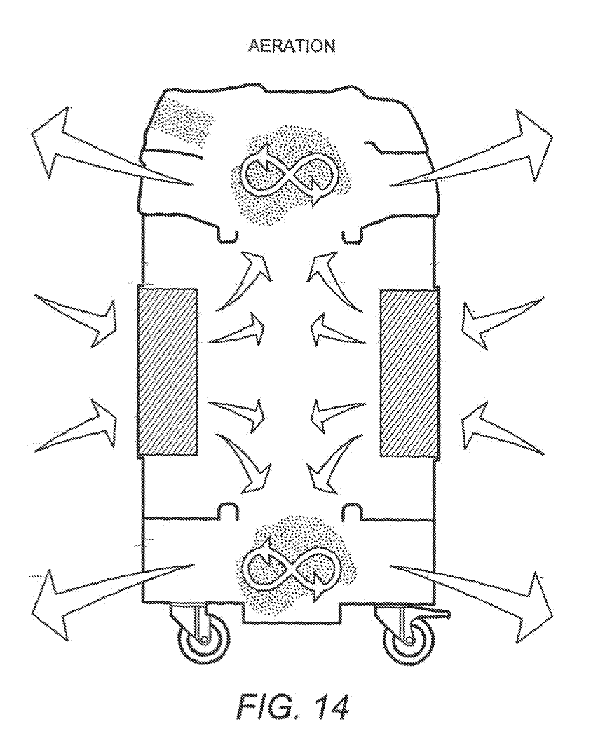

FIG. 14 is a similar view to FIG. 13 of the apparatus operating in a gas removal or aeration mode.

The invention relates to a combined sterilant vapour distribution and removal apparatus which is essentially a single piece of apparatus which combines means for the distribution of sterilant vapour in an enclosure during a gassing phase of the sterilisation process and means for removing the sterilant from the air in the enclosure during a subsequent aeration phase.

Referring to FIGS. 1 to 4 of the drawings, there is shown a first embodiment of the apparatus 10 of the present invention. The apparatus 10 comprises a chassis 11, preferably mounted on castor wheels 12 supported on swivel mountings 13 for ease of mobility of the apparatus around a room or other enclosure in which it is to be used.

The chassis 11 of the apparatus is preferably of a generally square configuration and carries a main housing 14 which is also generally square in plan view and is of generally upright form. The housing 14 is divided into an upper section 15 into which air/sterilant vapour from the enclosure is drawn in and is discharged at high velocity. There is provision in the section 15 for adding water vapour to the circulating air/sterilant vapour passing through the section to raise the humidity in the enclosure.

The apparatus has a lower section 16 through which the airborne sterilant vapour in the enclosure is drawn at the end of the decontamination process, the lower section 16 having sterilant removal means 44 for removal of the sterilant content of the air, as shown in FIGS. 5 and 6.

Handles 17 project from the upper section 15 of the housing 14 for manipulating the apparatus 10 in the enclosure.

In one construction of the apparatus 10 the housing 14 has a front wall 18, a rear wall 19 and side walls 20. As shown in FIGS. 5 and 6, the upper section 15 of the housing 14 is divided from the lower section 16 by an internal horizontal partition 21 and the upper section 15 is itself divided by a horizontal partition 22 into upper and lower chambers 23,24. The lower chamber 23 has a bank of inlet apertures 25 in the front wall of the housing 14 for air flow into the lower chamber 23. The upper chamber 24 has two large outlet nozzles 26 spaced apart horizontally on each of the front, side and rear walls 18,19,20 of the housing 14. The nozzles 26 of each pair are preferably set at slightly divergent angles with respect to each other.

A high speed fan 27 is mounted in an aperture in the partition 22, which divides the upper and lower chambers 23,24, to draw air/sterilant vapour from the enclosure through the inlets 25 into the lower chamber 23 and to direct the air/vapour into the upper chamber 24 to be discharged from the outlet nozzles 26 at high speed. This enhances the circulation and distribution of the sterilant vapour in the air throughout the enclosure.

In a modification to the apparatus 10 valve or other means may be incorporated in the upper chamber 24 of the top section 15 to enable the different sides of the apparatus 10 to be selectively controlled. For example discharge on one side only or selected sides may be provided.

A humidifying device 28 may also optionally be included, for example mounted on the partition 21. Such a device 28 may comprise a water container, a pump and a delivery conduit 30 which extends through the partition 22 into the upper chamber 24 of section 15 to supply water to a flash evaporator device mounted on the partition 21 to create a supply of water vapour in the flow of air/sterilant through the chamber 24 to be entrained in the flow and thereby delivered to the enclosure to raise the humidity of the air in the enclosure.

The lower section 16 of the housing is divided by a partition 40 into a lower chamber 41 and an upper chamber 42. The upper chamber 42 has banks of inlet apertures 43 opening into the side walls 20 of the housing for entry of air/sterilant vapour into the upper chamber 42. Sterilant removal means 44 (catalytic or other suitable means) are mounted behind the apertures 43. The sterilant removal means 44 may comprise a filter medium embodying a catalytic filter with which the sterilant vapour reacts to be decomposed into harmless constituents. In the case of hydrogen peroxide vapour, the peroxide is decomposed into water or water vapour and oxygen.

The lower chamber 41 of the lower section 16 has banks of outlet apertures 50 located on the front, rear and side walls of the housing 14 for the discharge of air and any residual sterilant vapour from the lower chamber 41 back to the enclosure. The partition 40 dividing the upper and lower chambers 41,42 has a large central aperture in which a high speed fan 51 is mounted for drawing sterilant vapour into the upper chamber 42 through the inlet apertures 43 and sterilant vapour removal means 44 and to discharge the air and any residual sterilant through the outlet apertures 50 in the lower chamber 41 of the lower section 16.

The partition 40 also carries a small axial flow pump 52 for drawing sterilant vapour from the enclosure during the decontamination phase of the enclosure through the apertures 50 to circulate sterilant vapour throughout the upper and lower chambers 41,42 of the lower section 16 of the housing 14 to ensure that the surfaces throughout those chambers 41,42 of the lower section 16 are sterile. Thus when air is drawn into the upper chamber 42 and the sterilant contained in the air is removed, the air returned from the lower section 16 to the enclosure is not contaminated by bacteria in the upper or lower chambers 41,42 of the lower section 16.

Reference is now made to FIGS. 7 to 9 which illustrate the apparatus 10 in its different modes as referred to above. In FIG. 7, the apparatus 10 is being operated to enhance circulation and distribution of sterilant vapour throughout the enclosure during a decontamination operation in the enclosure. Thus air/sterilant vapour is drawn into the lower chamber 23 of the upper section 15 and accelerated by the fan 27 between the upper and lower sections 23,24 and discharged at high speed through the nozzles 26 to enhance distribution of the vapour throughout the enclosure.

Furthermore the castor wheels 12 means that the apparatus 10 can be specifically oriented to direct the discharged stream of airborne sterilant vapour in one or more specific directions. As an alternative to the wheels 12, or in addition thereto, the apparatus may also include other means of orienting the apparatus 10 as a whole, or a part thereof, to selectively provide directionality to the discharged airborne vapour stream. For example the upper section 15 may be rotatable relative to the rest of the apparatus 10.

In the mode shown in FIG. 8, the humidifier is in operation to add further water vapour to the sterilant vapour/air being circulated to raise the humidity of the vapour content of the enclosure and thereby accelerate the decontamination process. The optional humidifier part of the unit may be controlled by the separate sterilant vapour generator or a control device of the apparatus 10, which monitors the relative humidity (RH) of the enclosure atmosphere. If the starting RH falls below a preset value then a control signal is generated to operate the humidifier during the decontamination process.

FIG. 9 shows the aeration function in operation in the lower section 16 of the housing 14 in addition to distribution in the upper section 15. Thus air containing sterilant vapour is drawn into the lower section 16 through the inlets 43 and through the sterilant vapour removal means 44 where the sterilant vapour is removed and the air, and any residual vapour not yet removed, is discharged from the lower chamber 41 of the lower section. During the aeration phase of the sterilisation cycle the high capacity fan 51, which is mounted in the base of the apparatus 10, moves the air by drawing it in through the inlets 43 and driving it through the sterilant removal means 44. The scrubbed air leaves the apparatus 10 through the outlet apertures 50 located at the base of the walls 18,19,20 of the housing 14.

The housing 14 preferably has means for connecting two units of apparatus 10 together, or one unit of apparatus and a vapour generator, so that two units can be linked together or a unit linked to a vapour generator for moving the units collectively. The connecting means may be in the form of a spaced hook shaped clips 48 mounted towards the lower end of the housing and the rear wall 19 has a horizontal bar 49 mounted towards the lower end of the housing 14.

The specific construction of the apparatus 10 can be modified from that described above and another embodiment of the apparatus 10 is shown in FIGS. 10 to 14. This apparatus has a number of similar features to the previously described apparatus 10 and the same reference numerals have been used to indicate the same parts.

One difference lies in the design of the housing 14 and its internal configuration. In the previously described embodiments aeration is effected by drawing air containing sterilant vapour into the lower section 16 through the inlets 43 and the air and any residual vapour not yet removed is discharged from the lower chamber 41 of the lower section 16 via outlet apertures 50 by the action of the fan 51. In the embodiment illustrated in FIGS. 10 to 14 the air is still drawn into the lower section 16 through the inlets 43, but the air flow is split and discharged through not only the outlet apertures 50, but also through the inlet apertures 25 in the upper section 15. This is illustrated clearly in FIG. 14. This can be achieved by using the axial fan 27, which in the previous embodiment was used only in the distribution phase of the apparatus 10.

For the distribution phase, one or more small high speed fans 60 may be located adjacent the outlet nozzles 26 (see FIGS. 12 and 13) which draws air/sterilant vapour into the upper section 15 via the inlet apertures 25 and discharges it at high speed through the outlet nozzles 26.

This arrangement enables the aeration and distribution phases to be totally operable independent of each other. Furthermore the use of common components for the distribution and aeration activity simplifies the construction, which leads to a saving in weight, costs and manufacturing time.

It will also be noted that this apparatus 10 has no humidifying device 28.

Other configurations can also be used, such as the use of a single axial fan in the upper section 15 which is used by both the distribution apparatus and the aeration apparatus. In such an arrangement a valve arrangement may be used, preferably in the lower section 16 in the housing 14. The valve(s) may be operated to open during the aeration phase so that air containing sterilant vapour is only drawn into the lower section 16 through the inlets 43 and directed back out through the outlet nozzles 26. During the distribution phase the valve(s) may be operated to close and to effectively seal off the filter(s) 44. Thus air/sterilant vapour is only drawn into the upper section 15 through the inlet apertures 25, 50 and discharged via the outlet nozzles 26.

In yet another arrangement the apparatus 10 may comprise a housing which incorporates the means for removing the sterilant from the air during the aeration phase, namely an axial fan mounted in the base of the unit, air inlets, filter or other means for removing the sterilant vapour from the air and air outlets. The means for the distribution of sterilant vapour in the enclosure during the gassing phase comprise a radially mounted distribution fan mounted on top of the unit which sucks in the surrounding air and blows it out to distribute it around the enclosure. This fan may be rotatably mounted to increase its effectiveness.

1. An apparatus, for use with apparatus for delivering airborne sterilant vapour to an enclosure for a sterilisation process, said apparatus comprising:
distribution means operable during a gassing phase of the sterilisation process to draw airborne sterilant vapour from the enclosure into the apparatus and to discharge and distribute said airborne sterilant vapour in the enclosure;
means to selectively control the discharge of sterilant vapour from different sides of the apparatus so that the sterilant vapour is discharged either on selected sides or one side only; and
means operable during an aeration phase to remove the sterilant from air in the enclosure.

2. An apparatus as claimed in claim 1, wherein the distribution means comprise air moving means for creating an air flow in the enclosure.

3. An apparatus as claimed in claim 2, wherein the distribution means comprise at least one inlet, at least one outlet and the air moving means comprise means for drawing air into the apparatus from outside the apparatus through the at least one inlet and discharging the air through the at least one outlet at an increased velocity.

4. An apparatus as claimed in claim 3, wherein the apparatus comprises a housing which has multiple inlets on one or more sides of the housing.

5. An apparatus as claimed in claim 4, wherein the housing has outlets on one or more sides of the housing and, in the case of more than one side, means are provided for selecting which sides are open for discharge.

6. An apparatus as claimed in claim 2, wherein the air moving means comprise a motor driven fan.

7. An apparatus as claimed in claim 1, wherein means are provided for delivering water vapour to the air flow to be incorporated in the air flow generated by distribution means.

8. An apparatus as claimed in claim 7, wherein the means for delivering water vapour to the air flow in the first chamber comprise a water supply and means for flash evaporating water from the supply into the flow of air.

9. An apparatus as claimed in claim 1, wherein the sterilant removal means comprise at least one inlet, means for removing sterilant vapour from the air, at least one outlet, and air moving means for drawing air into the apparatus through the inlets through the sterilant removal means and discharging the air through the outlets to thereby reduce the concentration of sterilant in the air outside the apparatus.

10. An apparatus as claimed in claim 9 in which at least one of the inlets of the distribution means is also an inlet of the sterilant removal means.

11. An apparatus as claimed in claim 9 in which at least one of the outlets of the distribution means is also an outlet of the sterilant removal means.

12. An apparatus as claimed in claim 9 in which the air moving means of the distribution means is the, or part of the, air moving means of the sterilant removal means.

13. An apparatus as claimed claim 9 wherein the means for removal of sterilant from the air flowing between the inlets and outlets comprise means located adjacent the inlets.

14. An apparatus as claimed in claim 9, wherein the air moving means of the sterilant removal means comprises a radial fan.

15. An apparatus as claimed in claim 9, wherein the direction of discharge of air through the outlets is selectable.

16. An apparatus as claimed claim 2, further comprising valve means operable to prevent the flow of air in through the at least one inlet of the sterilant removal means during operation of the distribution means.

17. An apparatus as claimed in claim 1, wherein the apparatus is a single unit.

18. An apparatus as claimed in claim 1, wherein the apparatus is mounted on wheels.

19. An apparatus as claimed in claim 1, wherein the apparatus has handles.

20. An apparatus as claimed in claim 1, wherein the apparatus has coupling means for linking the housing to a mobile apparatus for delivering sterilant vapour to an enclosure to enable the two forms of apparatus to be manoeuvred together.

21. (canceled)
